SEQUENCE LISTING (6 sequences)

SEQ ID NO: 1 - DNA, 306 bp, Zea mays
  CDS (1)...(306)
atggctttcgcgcccaaaacgtcctcctcctcctcgctctcctcggcgttgcaggcagctcagtcgccgccgctgctcctgaggcggatgtcgtcgaccgcaacaccgagacggaggtacgacgcggccgtcgtcgtcactaccaccaccactgctagagctgcggcggctgctgtcacggttcccgccgccccgccgcaggcgccggcgccgccgccggtgccaccaaagcaagcggcggcacccgccgagaggaggagccgtccggtgtcggacaccatggcggcgctcatggccaagggcaag

SEQ ID NO: 2 - protein, 102 aa, Zea mays
MAFAPKTSSSSSLSSALQAAQSPPLLLRRMSSTATPRRRYDAAVVVTTTTTARAAAAAVTVPAAPPQAPAPPPVPPKQAAAPAERRSRPVSDTMAALMAKGK

SEQ ID NO: 3 - DNA, 303 bp, Zea mays
  CDS (1)...(303)
atggctttcgcgcccaaaacgtcctcctcctcctcgctgtcctcggcgttgcaggcagctcagtcgccgccgctgctcctgaggcggatgtcgtcgaccgcaacaccgagacggaggtacgacgcggccgtcgtcgtcactaccaccaccactgctagagctgcggcggctgctgtcacggttcccgccgccccgccgcaggcgcgccgccgccgccggtgccaccaaagcaagcggcggcacccgcagaggaggagccgtccggtgtcggacaccatggcggcgctcatggccaagggcaag

SEQ ID NO: 4 - protein, 101 aa, Zea mays
MAFAPKTSSSSSLSSALQAAQSPPLLLRRMSSTATPRRRYDAAVVVTTTTTARAAAAAVTVPAAPPQARRRRRCHQSKRRHPQRRSRPVSDTMAALMAKGK

SEQ ID NO: 5 - DNA, 30 bp, Zea mays
gaggcccgctcttgctataaacgaggcagc

SEQ ID NO: 6 - DNA, 28 bp, Zea mays
ggatcgatctcggccggctagctagcag

(19) United States
(12) Patent Application Publication
(10) Pub. No.: US 2001/0052140 A1
(43) Pub. Date: Dec. 13, 2001

(54) Methods of selection and development of plants having improved root quality and root lodging resistance

(76) Inventor: Wesley B. Bruce, Urbandale, IA (US)

Correspondence Address: Debra L. Blair, Patent Counsel, 7100 N.W. 62nd Avenue, P.O. Box 1000, Johnston, IA, 50131-1000, US

(21) Appl. No.: 09/816,279

(22) Filed: Mar. 23, 2001

Related U.S. Application Data
(60) Provisional application No. 60/191,927, filed on Mar. 24, 2000.
(60) Provisional application No. 60/235,803, filed on Sep. 27, 2000.

(51) Int. Cl. C12N 15/29; A01H 5/00; C12N 15/82

(52) U.S. Cl. 800/320.1; 435/419; 800/279; 800/266; 800/267; 800/290; 536/23.6

(57) ABSTRACT

The invention provides methods for improving root quality and root lodging resistance in plants, as well as transformed plants exhibited improved root quality and root lodging resistance. The present invention provides methods and compositions relating to altering root quality and root lodging resistance in plants. The invention further provides recombinant expression cassettes, host cells, and transgenic plants.

### CROSS-REFERENCE TO RELATED APPLICATION

This application claims the benefit of, and hereby incorporates by reference, provisional patent application 60/191,927, filed Mar. 24, 2000, and provisional patent application 60/235,803, filed Sep. 27, 2000.

### TECHNICAL FIELD

The present invention relates generally to plant breeding and plant transformation. More specifically, it relates to improved methods for selecting and advancing breeding lines based on molecular predictors of a desired phenotype. The present invention further generally relates to plant molecular biology. More specifically, it relates to nucleic acids and methods for modulating their expression in plants.

### BACKGROUND OF THE INVENTION

Breeding programs strive to improve appropriate traits while countering negative environmental influences that reduce crop yield. Some agronomic characteristics, such as plant height or flowering time, are more easily bred; others, such as improved root-related traits, are usually selected indirectly. Since root lodging, a failure of plants to maintain an upright stature, can drastically reduce harvestable yield in numerous crops (e.g. corn, [Carter and Hudelson, 1988]), it is an important trait for breeding programs. While improving yield, attempts are made to apply selection pressure in improving root lodging resistance in hybrids, usually scored as percent of the plants root lodged. However, this measurement is difficult to reproduce due to the reliance on adverse weather conditions (e.g. high winds) to reveal contrasts in root lodging scores. Many studies have incorporated deliberate tests whereby hybrid and inbred lines with varying resistances to root lodging were scored following mechanical perturbances under a variety of field conditions (Beck et al., 1987; Guingo and Hébert, 1997; Kato and Koinuma, 1999). Also, the underlying genetic complexity observed with root lodging susceptibility complicates the selection for any one root morphological trait (Hébert et al., 1992), so combining individual traits may be needed for positive selection in breeding programs.

The relationship of root lodging with root and aerial morphological traits has been examined in numerous studies. Root mass, root volume, root numbers, diameter of individual roots, angle of root growth from the stem, stalk diameter, ear height to plant height ratios, and length of base internodes were all shown to correlate with either natural or artificial root lodging resistance (Baker et al., 1998; Crook et al., 1994; Ennos et al., 1993; Guingo and Hébert, 1997; Hébert et al., 1992; Kato and Koinuma, 1999; Seo et al., 1996; Stamp and Kiel, 1992). Another study demonstrated that soil components affect root development characteristics and that these traits correlate with root lodging (Goodman and Ennos, 1999). Based on such studies, it is clear that associating any one trait with resistance to root lodging is difficult. In fact, Guingo and Hébert (1997) demonstrated that combinations of at least three root traits—namely diameter, the number of roots on the upper-tiered nodes, and angle of root growth—correlated well with the stiffness coefficient, a measure of root lodging resistance (Guingo and Hébert, 1997). Remarkably, only a few studies on mapping quantitative trait loci (QTL) for maize root lodging per se exist, underscoring the difficulty of securing reproducible environmental conditions to measure this trait. However, QTL's for root morphological traits have been mapped in a few crop species (Guingo et al., 1998; Lebreton et al., 1995; McCouch and Doerge, 1995; O'Toole and Bland, 1987). These studies demonstrate the complexity of inheritable root traits that may provide the foundation for resistance to root lodging. Understanding the molecular mechanisms underlying the phenotypic expression of this trait would greatly benefit breeding programs.

Thus, there is a need to identify polynucleotides associated with improved root quality and root lodging resistance in maize using a combination of morphological and molecular techniques, providing advantages for breeding programs by reducing or eliminating reliance on environmental conditions for selection. The present invention provides polynucleotides associated with improved root quality and improved root lodging resistance, whereby plants having at least one, or more specifically a combination of two or more, of the polynucleotides exhibit improved root quality and root lodging characteristics. The present invention also provides methods for selecting and advancing breeding lines using these polynucleotides as molecular markers or primers, and methods of developing transformed plants whereby the regenerated plants exhibit improved root quality and improved root lodging resistance.

### SUMMARY OF THE INVENTION

Two related inbred lines of maize are found to show contrasting root traits and root lodging scores. These inbred lines are also extensively field-tested for root lodging as hybrids resulting from crosses with common tester parents. It is shown that certain genes are differentially expressed in the root tissues of these two lines. These genes directly or indirectly influence root development, thus affecting the plants' respective soil anchoring properties. To demonstrate this influence, root morphological traits are measured and expression profiling is conducted on whole root tissue from the two inbred lines using the mRNA profiling analysis, GeneCalling™ (see Bruce et al., 2000; Shimkets et al., 1999).

The GeneCalling™ analysis is an open-architecture, gel-based assay that reproducibly measures changes in the levels of tens of thousands of cDNA fragments (Bruce et al., 2000; Shimkets et al., 1999). This analysis provides a means of comparing cDNA fragment profiles from different RNA samples and connecting the cDNA fragments to genes that modulate in expression levels between treatments. The GeneCalling™ analysis has been successfully used to identify gene members of the known flavonoid pathway that were induced by appropriate transcription factors under inductive controls (Bruce et al., 2000). By this approach, several genes are identified whose differential expression between the two inbred lines demonstrate a role in root lodging resistance in maize.

Therefore, the present invention relates to methods to predict the level of root lodging resistance which a plant and its progeny will exhibit. The present invention also relates to methods for producing plants having improved root quality and improved root lodging resistance. The present invention also relates to transgenic plants having improved root quality and improved root lodging resistance.

### DETAILED DESCRIPTION OF THE FIGURES

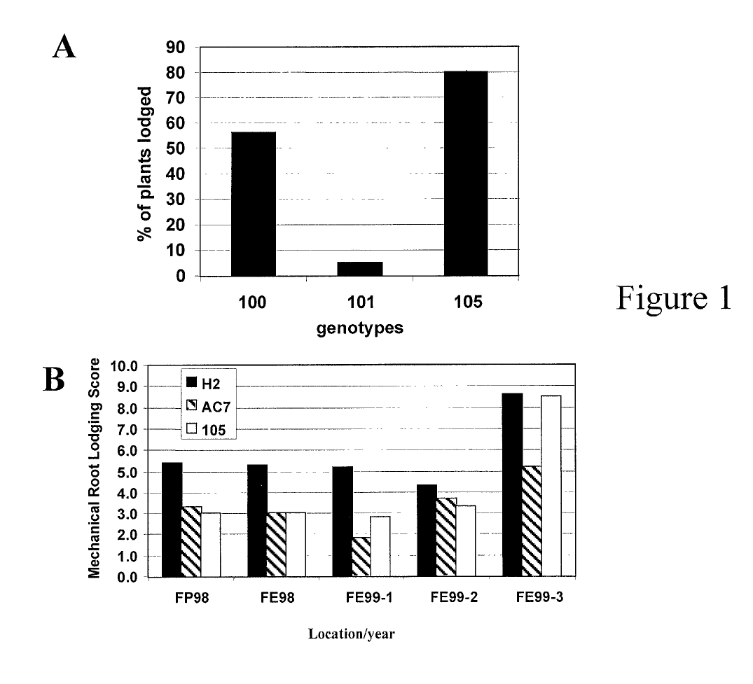

FIG. 1 is a bar graph showing differences in lodging among hybrids created using inbred lines 100, 101, and 105, and the inbred per se. (A) The percentage of plants lodged is shown for hybrids created using one of three inbred lines, 100, 101, and 105, and from five to twenty testers. Plants were scored for natural root lodging in either single-row or double-row plots per replicate. For inbred 100, n=247 replicates; 101, n=1401; 105, n=896. The standard deviation was 10% or less for the values shown. (B) Mechanical root lodging scores for a parental line, 105, used in an introgression backcross program, and two contrasting progeny lines which resulted, H2 and AC7. These lines were scored for resistance on a 1-9 scale where 1 is poor and 9 is excellent resistance. The standard error of the mean was 1.6 or less for the values shown.

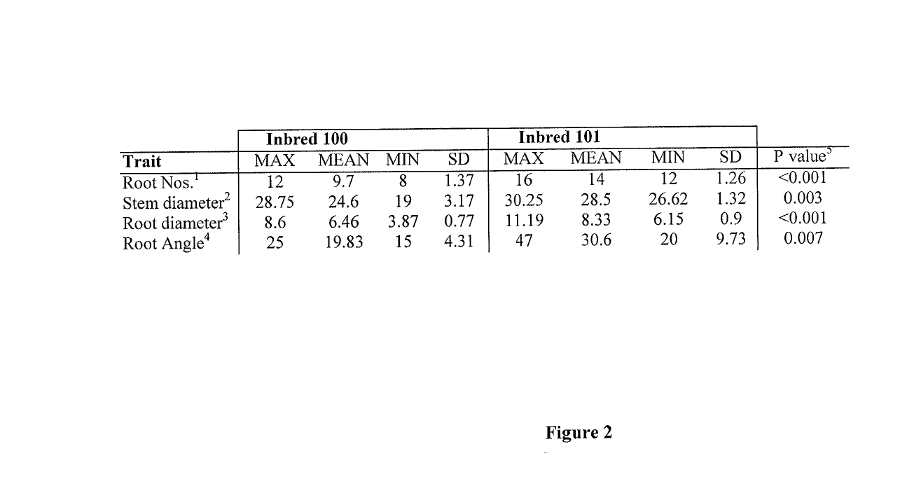

FIG. 2 is a table of selected morphological measurements for inbreds 100 and 101. “Root Nos.” is the number of roots on the seventh node. “Stem diameter” is the diameter, in mm, of the internode above the eighth root node. “Root diameter” is the average diameter, in mm, of five seventh-nodal roots per plant. “Root angle” is the angle of root growth, relative to the vertical, of five seventh-nodal roots per plant. The “P value” is based on a one-tailed z-test with α=0.01.

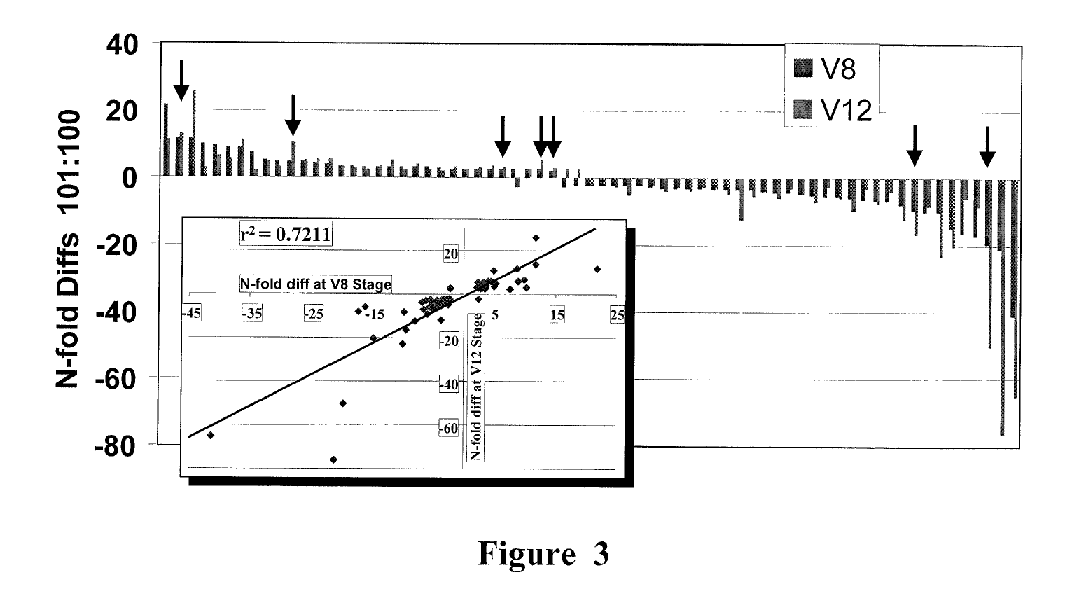

FIG. 3 includes a bar graph of the N-fold difference ratio (inbred 101:inbred 100) for 69 cDNA fragments at two developmental stages, and a linear regression of N-fold difference ratios at V8 against N-fold difference ratios at V12. The N-fold difference ratio (twofold or greater) of 69 cDNA fragments from inbred 101 compared to 100 from roots sampled at two developmental stages are shown and are from four to six replicate cDNA samples derived from two root samples. The standard deviation for each data point is less than 5% of the mean value.

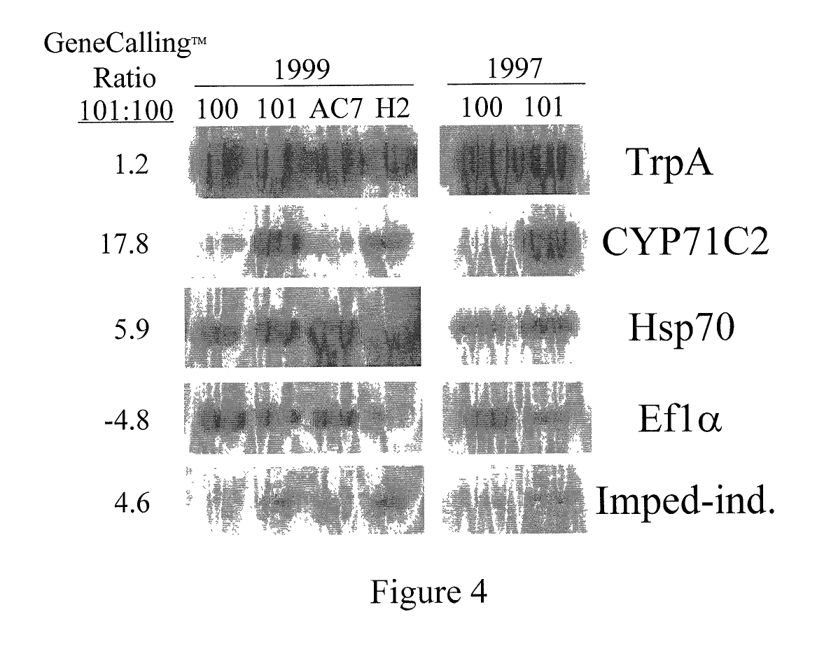

FIG. 4 shows the GeneCalling™ ratio and RNA gel blot analysis of the identified genes from V8 stage root samples from four maize lines. GeneCalling™-mediated ratio values are shown at the left and were calculated in the following manner: the proportion of the observed peak height value for a target cDNA fragment was first determined as the difference between the control peak value and the gene-specific-primer-competed peak value from the competitive PCR reactions. The final ratios are calculated as the competed PCR difference values from inbred 101 samples over inbred 100 samples. The year of sample harvest is shown above RNA gels. Gel blots for the V12 stage root samples showed little or no difference from those of the V8-stage root samples.

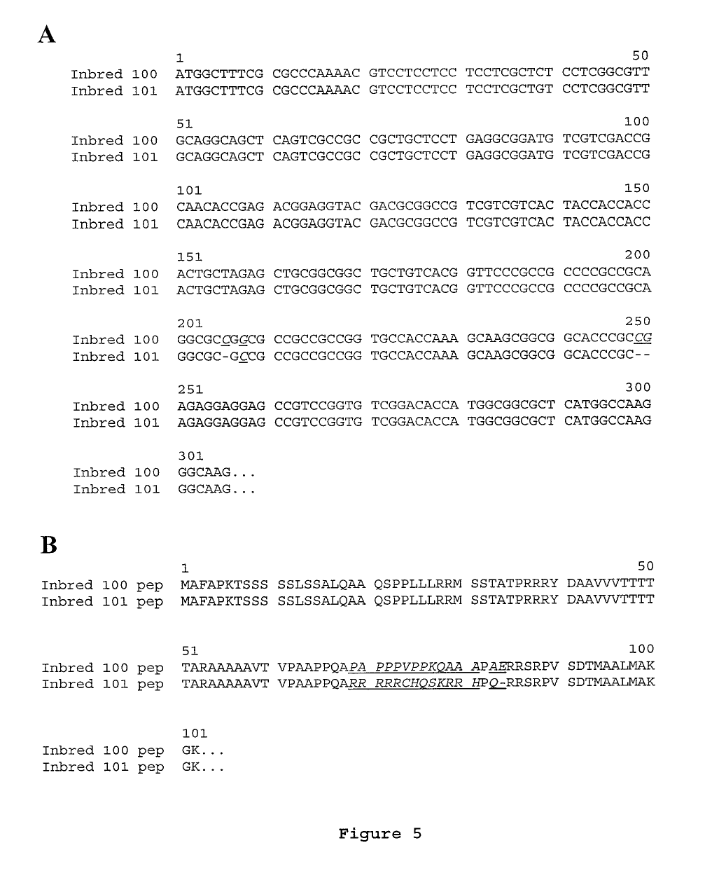

FIG. 5 is an alignment of nucleotide and amino acid sequences of the polymorphic N-terminal region of the TrpA gene from inbreds 100 and 101. (A) Nucleotide sequence alignment starting with the ATG and extending 305 nucleotides includes the three nucleotide polymorphism. (B) Amino acid alignment from the first methionine to amino acid 101. The underlined, italicized letters and the dashes denote the sequence divergence between inbred lines.

### DEFINITIONS

Unless otherwise provided, software, electrical, and electronics terms as used herein are as defined in The New IEEE Standard Dictionary of Electrical and Electronics Terms (5th edition, 1993). The terms defined below are more fully defined by reference to the specification as a whole. Section headings provided throughout the specification are not limitations to the various objects and embodiments of the present invention. Numeric ranges recited within the specification are inclusive of the numbers defining the range and include each integer within the defined range.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

An “expression profile” is the result of detecting a representative sample of expression products from a cell, tissue, or whole organism, or a representation (picture, graph, data table, database, etc.) thereof. For example, many RNA expression products of a cell or tissue can be simultaneously detected on a nucleic acid array, or by the technique of differential display or modification thereof such as Curagen's GeneCalling™ technology. A “portion” or “subportion” of an expression profile, or a “partial profile” is a subset of the data provided by the complete profile, such as the information provided by a subset of the total number of detected expression products.

The term “correlation”, unless indicated otherwise, is used herein to indicate that a “statistical association” exists between, for example, an expression product and the degree of root lodging resistance.

The phrase “hybrid plants” refers to plants which result from a cross between genetically different individuals.

As used herein, “nucleic acid” includes reference to a deoxyribonucleotide or ribonucleotide polymer, or chimeras thereof, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism, tissue, or of a cell type from that organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2nd ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein, the term “plant” includes reference to whole plants, plant parts or organs (e.g., leaves, stems, roots, etc.), plant cells, seeds and progeny of same. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention includes both monocotyledonous and dicotyledonous plants. A particularly preferred plant is Zea mays.

As used herein, “polynucleotide” includes reference to a deoxyribopolynucleotide, ribopolynucleotide, or chimeras or analogs thereof that have the essential nature of a natural deoxyribo- or ribo-nucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences, and to the substantial exclusion of non-target nucleic acids. Selectively hybridizing sequences typically have about at least 80% sequence identity, preferably 90% sequence identity, and most preferably 100% sequence identity (i.e., are complementary) with each other.

The phrase “tester parent” refers to a parent that is genetically different from a set of lines to which it is crossed. The cross is for purposes of evaluating differences among the lines. Using a tester parent in a sexual cross allows one of skill in the art to determine the genetic differences between the tested lines as to the phenotypic trait under consideration.

### DETAILED DESCRIPTION OF THE INVENTION

A. Overview

The present invention provides methods for predicting root lodging resistance. Maize (Zea mays L.) inbreds 100 and 101 originated from an F3 pool generated from a segregating F2 population of a cross between two Pioneer proprietary elite inbreds. Two plants were selfed six generations before undergoing trait evaluations. The inbreds 100 and 101 were analyzed with 106 RFLP, isoenzyme, and SSR markers, essentially as described in Beavis et al. (1994). Eighty of the 106 markers were identical between inbreds 100 and 101, suggesting that the genomes of these two lines were 75% homologous. Regions of marker differences were distributed throughout the genome.

Root lodging and morphology

Inbred lines 100 and 101 were crossed to several common testers, and the resulting F1 hybrids were evaluated in single- or two-row plots at a variety of locations in North America and Europe. Agronomic trait data such as grain yield and percent lodging were collected from the hybrids grown between 1993-1995. For root lodging resistance, the number of replicates examined exceeded 200. Root lodging scores were determined as a percent of plants lodged per replicate. FIG. 1A shows the average percentage of lodged plants per replicate for hybrids created by crossing the inbred lines to five to twenty testers. Inbred 100 produced hybrids with a significantly higher percentage of root lodging than hybrids from inbred 101. It is evident that the root-lodging-resistant phenotype can be manifested in heterozygous genotypes from a number of tester backgrounds.

The H2 and AC7 lines were generated from an introgression backcrossing program for root lodging resistance into the parental inbred 105 and showed contrasting root lodging scores (P. Desbons and S. Openshaw, unpublished results). During 1998 and 1999 at two European locations (designated “FP” and “E”, followed by the year tested), mechanical root lodging data were collected for the lines 105, H2 and AC7 either directly (“FP98”, “FE98” and “FE99-1”) or in hybrid combinations with two different testers (“FE99-2” and “FE99-3”) as shown in FIG. 1B. Since there was a lack of sufficient wind-damage, natural root lodging could not be measured. Ten to twelve plants per replicate for three replicates per location were pushed by hand using a one-meter wooden rod placed just below the primary ear. The plants were evaluated just after extensive irrigation. In 1999 the H2 and AC7 lines were grown in the same location as inbreds 100 and 101 for tissue harvesting and RNA gel blot analysis as described below.

The differences in root lodging scores between hybrids from inbreds 100 and 101 may be due in part to differences in root morphological characteristics in the parental inbreds, similar to what was observed by Guingo and Hébert (1997). FIG. 2 shows the root and stem characteristics for both inbreds averaged over the two years. Based on analysis of variance, the root morphology for each line did not vary significantly (P>0.63) between the two years of measurements, in contrast to observations made by Hébert et al. (1992). The seventh node was chosen for measurements based on the conclusion that upper nodal roots correlated more strongly with the strength of plant anchorage than did lower, older roots (Guingo and Hébert, 1997). Inbred 101 showed nearly a 30% increase in the number and diameter of 7th nodal roots over inbred 100. The angle of root growth from the vertical axis for inbred 101 was nearly 50% more than that for inbred 100, showing a mean value of 30.6°±9.73 (FIG. 2). These data support the observations that higher root numbers, thicker roots and larger-angled root systems correlated significantly with greater strength of plant anchorage (Ennos et al., 1993; Guingo and Hébert, 1997; Hébert et al., 1992).

Profiling of mRNA

To identify the genes that were specifically expressed in the roots of either inbred line, inbred RNA samples were subjected to GeneCalling™ analysis (see U.S. Pat. No. 5,871,697, issued Feb. 16, 1999, herein incorporated by reference; see also Bruce et al., 2000, and Shimkets et al., 1999). RNA samples were harvested from two stages prior to flowering, the V8 and V12 stages. A divergence in overall root traits is generally manifested between the V6 and V10 stage of development for inbreds 100 and 101. Additionally, for most maize lines, the upper nodal roots (5th-8th) initiate around the V8 stage and are essentially developed by the V15 stage (Ritchie et al., 1997), and these roots were suggested to play a major role in resistance to root lodging (Guingo and Hébert, 1997). Therefore, it was anticipated that any actively-transcribed genes contributing to differences in root anchorage would be evident within the V8-V12 developmental stages.

The GeneCalling™ analysis involved the quantitative comparisons of restriction enzyme-digested cDNA generated from whole root tissue of the two contrasting inbreds at the two developmental stages. This approach comprehensively samples cDNA populations in a highly sensitive manner, allowing for detection of both expression levels and restriction fragment length polymorphisms (RFLP) associated with individual cDNA fragments. In root RNA samples from both inbred lines, 13,630 and 13,544 cDNA fragments were detected at the V8 and V12 developmental stages, respectively. Comparing the cDNA fragment trace data between inbreds, 229 and 325 cDNA fragments showed two-fold or greater differences in the V8 and V12 developmental stages, respectively. Only cDNA fragments showing highly significant gel trace differences (P<0.01) were included in the analysis.

Comparing gel trace data of the cDNA fragments showing twofold or greater difference between the inbred lines revealed 69 cDNA fragments in common for both developmental stages, as shown in FIG. 3. There was also a good correlation in N-fold difference between the two developmental stages (FIG. 3, inset graph). These 69 cDNA fragments were therefore predicted to correspond to genes which relate to overall architectural differences between the inbreds, rather than those which respond to environmental fluctuations occurring between the two developmental stages.

About half of these 69 cDNA fragments were expressed at higher levels in inbred 101 than in inbred 100. Seven of the 69 cDNA fragments were selected for further analysis. Five cDNA fragments were used for direct competitive PCR confirmations with three publicly known sequences, while the remaining two were cloned, sequenced and confirmed by competitive PCR. The oligonucleotide primers designed for these public genes used in the competitive PCR reaction successfully competed for the original cDNA fragment amplification.

The seven fragments corresponded to five known genes, tryptophan synthase (TrpA; (Kramer and Koziel, 1995; GenBank Accession No. X76713)), heat shock protein 70 (Hsp70; Rochester et al., 1986; GenBank Accession Nos. X03714, X03697, X03658), elongation factor 1α (Ef1α; Cao et al., 1997; GenBank Accession No. U76259), cytochrome P450-dependent monooxygenase (CYP71C2; Frey et al., 1995, GenBank Accession No. X81829; Frey et al., 1997, GenBank Accession No. Y11404), and an impedance-induced protein (Huang et al., 1998; GenBank Accession Nos. AF001634, AF001635). Three cDNA fragments corresponded to the TrpA gene, two of which were part of the original seven fragments targeted for further analysis.

The competitive PCR reaction provides a sensitive method for determining the proportion of a target cDNA fragment that is present in a band within the gel trace data, and it can also readily reveal intra-fragment polymorphisms that exist between different genotypes. The cDNA fragment for TrpA was first detected with an apparent 17-fold higher expression level in inbred 101 than in 100. However, based on the competitive PCR reaction with both inbred samples, this difference was due to a polymorphism. The gel trace data revealed that three TrpA gene cDNA fragments from inbred 101 were approximately nine (±1.5) nucleotides shorter than the corresponding fragments for inbred 100. All three fragments overlapped the same region in a portion of the 3′- translated and 3′-untranslated region of the transcript sequence. There was little change in the trace peak height values for the three fragments, suggesting small or no difference in TrpA expression levels between the inbred lines. The calculated ratio was 1.2 between inbred 101 to inbred 100, suggesting no difference in expression levels between the inbreds, as confirmed by the RNA gel blot analysis (FIG. 4).

To further investigate the potential allelic TrpA gene differences between inbreds 100 and 101, a genomic fragment from each inbred line was cloned and sequenced. Genomic DNA was isolated from root tissue of the inbreds 100 and 101 (Doyle and Doyle, 1990) and used in a PCR reaction with the Hot Star Taq Polymerase kit (Qiagen, Valencia, Calif.) according to the manufacturer's protocol. Primers (SEQ ID. Nos. 5 and 6) were used to amplify at least three independent identical fragments of the maize TrpA gene (Kramer and Koziel, 1995). The resulting fragments were cloned using the pCR2.1-TOPO kit (Invitrogen, Carlsbad, Calif.), sequenced to 4X coverage and aligned using the PC software of Sequencher 4.05 (Gene Codes Corp., Ann Arbor, Mich.).

FIG. 5 shows the alignment of the TrpA gene between the two inbreds 100 and 101 at both the nucleotide (SEQ ID Nos. 1 and 3, respectively) and amino acid (SEQ ID Nos. 2 and 4, respectively) levels. The TrpA of inbred 100 shows a three-base insertion relative to that of inbred 101, resulting in a change in 15 amino acids in the N-terminal region of the gene product. Also the TrpA gene was mapped to a locus on chromosome 1L (Davis et al., 1999) very close to where there is a polymorphic marker between the two inbred lines, confirming the differences detected both by GeneCalling™ and sequencing.

Contrasting levels of gene expression between inbred samples for four cDNA fragments were observed. However, no evidence of polymorphisms for these cDNA fragments was detected. These results do not rule out the possibility of polymorphisms affecting the restriction enzyme sites used in fragmenting the cDNA samples. Therefore, the cDNA fragments were examined further by RNA gel blot analysis, shown in FIG. 4, to confirm the expression differences detected by GeneCalling™. RNA gel blot analysis has been previously demonstrated to correlate well with the modulations of the levels of cDNA fragments detected by GeneCalling™ (Bruce et al., 2000). The differentially expressed cDNA fragments identified for the inbreds 100 and 101 by competitive PCR method were matched to corresponding EST clones from the Pioneer/DuPont EST collection and these clones were used as probes for the RNA gel blot analysis.

Two of the differentially-expressed genes identified by GeneCalling™, CYP71C2 and the impedance-induced-protein, were much more abundant in root tissues of two maize lines with high root lodging resistance (101 and H2) as compared to those with low resistance, as confirmed by RNA gel blot analysis (FIG. 4).

The CYP71C2 (also known as Bx3) gene product is a cytochrome P450-dependent monooxygenase, involved in the 2,4-dihydroxy-7-methoxy-1,4-benzoxazin-3-one (DIMBOA) synthetic pathway in maize (Frey et al., 1995; Frey et al., 1997). The CYP71C2 enzyme is part of a pathway of converting indole-3-glycerol phosphate into DIMBOA, and the indole-3-glycerol phosphate is an important intermediate for numerous secondary metabolic pathways, including tryptophan and indole acetic acid biosynthesis (Frey et al., 1997). Interestingly, the first committed enzyme in the DIMBOA pathway is Bx1, involved in indole production, and is highly homologous to the TrpA gene (Frey et al., 1997), suggesting an association between expression differences of members of the DIMBOA pathway including CYP71C2 and differences between the inbreds showing contrasting root traits.

Rutherford et al. (1998) demonstrated that mutations in the Arabidopsis TrpA gene (trp3-1) caused greater compressions in the root waving phenotype on tilted agar surfaces. The root waving phenomenon may be a result of integrating gravitropic and impedance avoidance stimuli with circumnutation-like growth. Rutherford et al. (1998) postulated that localized reduction in free L-tryptophan in the roots affected root tip rotation and the circumnutation-like growth in a gravitropism-independent manner. It is possible that maize genotypes showing differential resistance to root lodging may be producing varied levels of tryptophan-related metabolic enzyme activities (TrpA and CYP71C2) in root tissues, ultimately affecting root growth and architecture.

The impedance-induced gene was previously shown to be rapidly induced when elongating roots encounter physical impedance (Huang et al., 1998). This gene product is very similar to Zrp3, which is expressed in a subset of cortical cells near the expansion zone of root tips (John et al., 1992) and may be part of a multigene family in maize. The impedance-induced gene product may also function in the early stages of stress-inducible responses (Huang et al., 1998) and act as an indicator of the state of root growth under non-ideal soil conditions, especially for the contrasting maize lines.

Two genes, Ef1α and Hsp70, were identified by GeneCalling™ analysis as showing five to sixfold differences between the two inbreds, not entirely agreeing with differences detected in the RNA gel blot analysis. Fragment size modulations were not detected in the PCR confirmations for either of these genes, reducing the possibility of polymorphic differences. Since both genes belong to very highly conserved multigene families (Rochester et al., 1986; Bates et al., 1994; Berberich et al., 1995) and because the detection of cDNA-fragment-gel-trace differences is more gene specific than RNA gel blot analysis, cross hybridization may be occurring in the RNA gel blot analysis.

Based on the GeneCalling™ data, a Hsp70 gene family member was associated with differences in root morphology or root lodging. Hsp70 proteins have been shown to interact with DnaJ proteins via the DnaJ's “J-domain” (Zuber, 1998). One plant DnaJ protein was recently described as being encoded by the ARG1 gene. When this gene is mutated in Arabidopsis, altered gravitropic responses in roots and hypocotyl were observed without pleiotropic phenotypes (Sedbrook et al., 1999). The Hsp70 gene product showing differential expression in the two maize inbreds may influence gravitropic responses via a protein-protein interaction with the gene product of an orthologous maize ARG1. This interaction may generate differences in the angle of root growth that are important in root lodging resistance.

RNA from a second pair of maize lines (H2 and AC7) derived from a backcrossing program of introgressing root lodging resistance into a root lodging susceptible parent (inbred 105) was also used in the RNA gel blot analysis (FIG. 4). H2 exhibits significantly higher root lodging resistance relative to a control line, AC7, either directly or in one of the two hybrid combinations tested. (FIG. 1B). Since both the H2 and AC7 lines were grown in the same location a few rows away from inbreds 100 and 101 for RNA sampling in 1999, it is expected that all four lines experienced the same environmental influences. Four of the five genes tested showed RNA patterns similar to inbred 100 and 101, while the Hsp70 gene showed a converse pattern. At least for most of the identified genes, these data help confirm the expression differences observed with inbreds 100 and 101.

B. Exemplary Utility of the Present Invention

The present invention provides utility in such exemplary applications as screening breeding lines for resistance to root lodging and/or root quality using polynucleotides associated with root lodging resistance and/or improved root quality as molecular markers, PCR primers or other molecular techniques known to those of skill in the art. Measuring morphological traits provides an advantage in consistency of evaluation. Molecular characterization provides an advantage in that the high-throughput nature of profiling can dramatically speed the process of selection and increase the rate of crop improvement.

The present invention also provides utility through methods of transforming plant tissue and regenerating transformed plant tissue into plants comprising the polynucleotides or the genes associated with the polynucleotides of the present invention, whereby the transformed plants exhibit improved root quality and/or improved root lodging resistance. A further utility is the transformation of plants with a combination of two or more of the polynucleotides of the present invention whereby the transformed plants exhibit improved root quality and/or improved root lodging resistance.

C. Exemplary Preferable Embodiments

While the various preferred embodiments are disclosed throughout the specification, exemplary preferable embodiments include the following:

(i) Root morphology analysis. Whole roots are carefully excavated from the two inbred lines at the V8 developmental stage. Soil is removed by two gentle washes in water and roots are patted dry with paper towels. The maize vegetative developmental stage designations are as previously described (Ritchie et al., 1997) and are dependent on the emergence of the leaf ligule (e.g. V8 refers to the emergence of the 8th ligule on the plant). Morphological measurements are taken essentially as described by Guingo and Hébert (1997). The number of roots on the seventh node (top most node of the incipient root development) and the diameter of five randomly chosen 7th nodal roots per plant are measured using an electronic caliper (Fred V. Fowler Co., Inc., Newton, Mass.). The angle of 7th nodal root growth from the vertical surface is measured on five roots per plants using a protractor. Stem diameter of the internode above the eighth node where adventitious roots emerge is measured. At least six plants per replicate are recorded. These data are averaged across years.

(ii) RNA isolation and RNA gel blot analysis. Whole roots are carefully excavated from the two inbred lines at the V8 and V12 developmental stages. Soil is removed by two gentle washes in water and roots are patted dry with paper towels. Within ten minutes of excavation, roots from two plants per replicate are frozen in liquid nitrogen in 50 mL conical tubes using a CP300 cryoshipper (Taylor-Wharton, Theodore, Ala.) for subsequent RNA isolation. Total RNA is isolated from V8- and V12-stage whole roots using a protocol previously described (Dehesh et al., 1990). RNA gel blot analysis is conducted using 10 μg of total RNA per gel lane as described by Bruce et al. (2000). The blots may be successively probed and re-stripped using the Strip-EZ kit (Ambion, Inc.) according to manufacturer's protocol, using randomly-primed ³²P-labeled probes generated from the Pioneer/DuPont maize expressed sequence tag (EST) database representing genes of interest, such as Ef1α (cssaq52), TrpA (czaal73), Hsp70 (cgeuk42), impedance-induced protein (crtba20) and CYP71C2 (cebae55).

(iii) Recombinant methods for constructing nucleic acids. The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes which selectively hybridize, under stringent conditions, to the polynucleotides of the present invention are used to identify the desired sequence in a cDNA or genomic DNA library. Isolation of RNA, and construction of cDNA and genomic libraries is well known to those of ordinary skill in the art. See, e.g., Plant Molecular Biology: A Laboratory Manual, Clark, Ed., Springer-Verlag, Berlin (1997); and, Current Protocols in Molecular Biology, Ausubel, et al., Eds., Greene Publishing and Wiley-Interscience, New York (1995).

(iv) Synthetic methods for constructing nucleic acids. The isolated nucleic acids of the present invention can also be prepared by direct chemical synthesis by methods such as the phosphotriester method of Narang et al., Meth. Enzymol. 68: 90-99 (1979); the phosphodiester method of Brown et al., Meth. Enzymol. 68: 109-151 (1979); the diethylphosphoramidite method of Beaucage et al., Tetra. Lett. 22: 1859-1862 (1981); the solid phase phosphoramidite triester method described by Beaucage and Caruthers, Tetra. Letts. 22(20): 1859-1862 (1981), e.g., using an automated synthesizer, e.g., as described in Needham-VanDevanter et al., Nucleic Acids Res., 12: 6159-6168 (1984); and, the solid support method of U.S. Pat. No. 4,458,066. Chemical synthesis generally produces a single-stranded oligonucleotide. This may be converted into double-stranded DNA by hybridization with a complementary sequence, or by polymerization with a DNA polymerase using the single strand as a template. One of skill will recognize that while chemical synthesis of DNA is best employed for sequences of about 100 bases or less, longer sequences may be obtained by the ligation of shorter sequences.

(v) Detection of nucleic acids. The present invention further provides methods for detecting a polynucleotide of the present invention in a nucleic acid sample suspected of containing a polynucleotide of the present invention, such as a plant cell lysate, particularly a lysate of maize. The nucleic acid sample is contacted with the polynucleotide to form a hybridization complex. The polynucleotide hybridizes under stringent conditions to a gene encoding a polypeptide of the present invention. Detection of the hybridization complex can be achieved using any number of well-known methods. For example, the nucleic acid sample, or a portion thereof, may be assayed by hybridization formats including but not limited to, solution phase, solid phase, mixed phase, or in situ hybridization assays.

Detectable labels suitable for use in the present invention include any composition detectable by spectroscopic, radioisotopic, photochemical, biochemical, immunochemical, electrical, optical or chemical means. Useful labels in the present invention include biotin for staining with labeled streptavidin conjugate, magnetic beads, fluorescent dyes, radiolabels, enzymes, and colorimetric labels. Other labels include ligands which bind to antibodies labeled with fluorophores, chemiluminescent agents, and enzymes. Labeling the nucleic acids of the present invention is readily achieved, such as with labeled PCR primers.

Although the present invention has been described in some detail by way of illustration and example for purposes of clarity of understanding, it will be obvious that certain changes and modifications may be practiced within the scope of the appended claims.

### EXAMPLES

### Example 1

This example describes development of inbreds and hybrids and scoring for root lodging.

Pioneer Zea mays L. inbreds 100 and 101 originated from an F3 pool generated from a segregating F2 population of Pioneer proprietary elite lines. Two plants were selfed six generations before undergoing trait evaluations. The inbred 100 and 101 lines were analyzed by 106 RFLP, isoenzyme and SSR markers essentially as described (Beavis et al., 1994). Generally, these inbred lines were crossed to several common testers and the resulting F1 hybrids were evaluated in single- or two-row plots at a variety of locations in North America and Europe. Agronomic trait data such as grain yield and percent lodging were collected from such hybrids grown between 1993-1995. For root lodging resistance, the number of replicates examined exceeded 200 where indicated. Root lodging scores were determined as a percent of plants lodged per replicate. The H2 and AC7 lines were generated from an introgression backcrossing program for root lodging resistance into the parental inbred line 105 and showed contrasting root lodging scores. The H2 and AC7 lines were grown in the same location as inbreds 100 and 101 in 1999 for tissue harvesting as described below.

### Example 2

This example describes GeneCalling™ analysis.

GeneCalling™ analysis was performed according to Shimkets et al. (1999), except that poly(A)-enriched RNA was isolated from 50 μg of total RNA and at least 41 restriction enzyme pairs were used. The GeneCalling™ gel trace data from four to six replicate cDNA samples derived from two plants per inbred line for the inbred 101 were compared to those of inbred 100 for both developmental stages. RNA was purified, converted to cDNA and fragmented using pairs of restriction enzymes. Adapters were then ligated to the ends of the fragments and PCR amplified. Since one of the PCR primers was labeled with a fluorescent tag, fluorescamine (FAM), amplified fragments were visualized during electrophoresis. For each restriction enzyme-pair reaction per sample, electronic images of gel lane traces were collected and placed in a sample trace database. Comparisons of the trace databases revealed specific expression differences that were characterized by length of the amplified fragment and restriction enzyme sequence information.

The identity of each differentially expressed gene fragment was established either by a GeneCalling™ search in a sequence database, or by cloning and sequencing the desired cDNA fragment. The identity of the cDNA fragment was confirmed by competitive PCR in which the original PCR reaction was re-amplified in the presence or absence of an excess of an unlabeled, gene-specific PCR primer. Further characterization of known and novel sequences, identified in the GeneCalling™ analysis as differentially expressed, was obtained by BLASTX and BLASTN analysis (Altschul et al., 1990) against public and proprietary databases. Those with little or no match to public databases having an expected probability value less than 1.0×10⁻⁵ were subjected to BLAST 2.0 searches in the Pioneer/DuPont maize EST database. EST sequences that matched a novel cDNA fragment sequence with significant similarities were then used in BLASTX searches to determine likely gene identities.

### Example 3

This example describes use of RNA gel blot analysis to predict root lodging resistance among maize inbreds and hybrids. Maize inbred or hybrid RNA samples harvested from the V8 or V12 stages are subjected to analysis. Total RNA is extracted from whole root tissue using the method of Dehesh et al., 1990. Isolated from total RNA with the use of a PolyATtract® kit (Promega), approximately 2 μg of poly(A)-enriched RNA is separated on a 1.2% SeaKem gel containing MOPS (Ambion, Inc.) and 4% formaldehyde. After electrophoresis, the gel is washed twice in 2×SSC (1×SSC is 0.15 M NaCl and 0.015 M sodium citrate) and blotted overnight onto a Nytran membrane by using the TurboBlotter (Schleicher and Schuell) system and protocol. The blot is air-dried for 15 minutes and UV cross-linked in a Stratalinker (Stratagene) at 1200 μJ/cm². The RNA gel blot is prehybridized and hybridized in ExpressHyb buffer (Clontech), according to the manufacturer's protocol, with randomly primed ³²P-labeled probes from the Pioneer/DuPont EST collection which correspond to the four differentially-expressed cDNA fragments (CYP71C2, Hsp70, Ef1α, and impedance-induced) identified herein for the inbreds 100 and 101. The blot may be successively probed and stripped by using the Strip-EZ kit (Ambion, Inc.) according to the manufacturer's protocol. After probing, the blot is exposed to X-ray film for one to four days. The resulting expression profiles are compared to the results shown in FIG. 4, and root-lodging-resistant inbreds are selected based on similarity to root-lodging-resistant inbreds 101 and H2.

### Example 4

This example describes the construction of a cDNA library.

Total RNA can be isolated from maize tissues with TRIzol Reagent (Life Technology Inc. Gaithersburg, Md.) using a modification of the guanidine isothiocyanate/acid-phenol procedure described by Chomczynski and Sacchi (Chomczynski, P., and Sacchi, N. Anal. Biochem. 162, 156 (1987)). In brief, plant tissue samples are pulverized in liquid nitrogen before the addition of the TRIzol Reagent, and then further homogenized with a mortar and pestle. Addition of chloroform followed by centrifugation is conducted for separation of an aqueous phase and an organic phase. The total RNA is recovered by precipitation with isopropyl alcohol from the aqueous phase. The selection of poly(A)+RNA from total RNA can be performed using the PolyATtract® system (Promega Corporation, Madison, Wis.). Biotinylated oligo(dT) primers are used to hybridize to the 3′ poly(A) tails on mRNA. The hybrids are captured using streptavidin coupled to paramagnetic particles and a magnetic separation stand. The mRNA is then washed at high stringency conditions and eluted by RNase-free deionized water.

cDNA synthesis and construction of unidirectional cDNA libraries can be accomplished using the SuperScript Plasmid System (Life Technology Inc. Gaithersburg, Md.). The first strand of cDNA is synthesized by priming an oligo(dT) primer containing a Not I site. The reaction is catalyzed by SuperScript Reverse Transcriptase II at 45° C. The second strand of cDNA is labeled with alpha-³²P-dCTP and a portion of the reaction analyzed by agarose gel electrophoresis to determine cDNA sizes. cDNA molecules smaller than 500 base pairs and unligated adapters are removed by Sephacryl-S400 chromatography. The selected cDNA molecules are ligated into pSPORT1 vector in between of Not I and Sal I sites.

Alternatively, cDNA libraries can be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

### Example 5

This method describes construction of a full-length enriched cDNA library.

An enriched full-length cDNA library can be constructed using one of two variations of the method of Carninci et al. Genomics 37: 327-336, 1996. These variations are based on chemical introduction of a biotin group into the diol residue of the 5′ cap structure of eukaryotic mRNA to select full-length first strand cDNA. The selection occurs by trapping the biotin residue at the cap sites using streptavidin-coated magnetic beads followed by RNase I treatment to eliminate incompletely synthesized cDNAs. Second strand cDNA is synthesized using established procedures such as those provided in Life Technologies' (Rockville, Md.) “SuperScript Plasmid System for cDNA Synthesis and Plasmid Cloning” kit. Libraries made by this method have been shown to contain 50% to 70% full-length cDNAs.

The first strand synthesis methods are detailed below. An asterisk denotes that the reagent was obtained from Life Technologies, Inc.

A. First strand cDNA synthesis method 1 (with trehalose)

| | |
|---|---|
| mRNA(10 ug) | 25 μl |
| *Not I primer (5 ug) | 10 μl |
| *5× 1st strand buffer | 43 μl |
| *0.1 m DTT | 20 μl |
| *dNTP mix 10 mm | 10 μl |
| BSA 10 ug/μl | 1 μl |
| Trehalose (saturated) | 59.2 μl |
| RNase inhibitor (Promega) | 1.8 μl |
| *Superscript II RT 200 u/μl | 20 μl |
| 100% glycerol | 18 μl |
| Water | 7 μl |

The mRNA and Not I primer are mixed and denatured at 65° C. for 10 min. They are then chilled on ice and other components added to the tube. Incubation is at 45° C. for 2 min. Twenty microliters of RT (reverse transcriptase) is added to the reaction and start program on the thermocycler (MJ Research, Waltham, Mass.):

| | |
|---|---|
| Step 1 | 45° C. 10 min |
| Step 2 | 45° C. −0.3° C./cycle, 2 seconds/cycle |
| Step 3 | go to 2 for 33 cycles |
| Step 4 | 35° C. 5 min |
| Step 5 | 45° C. 5 min |
| Step 6 | 45° C. 0.2° C./cycle, 1 sec/cycle |
| Step 7 | go to 7 for 49 cycles |
| Step 8 | 55° C. 0.1° C./cycle, 12 sec/cycle |
| Step 9 | go to 8 for 49 cycles |
| Step 10 | 55° C. 2 min |
| Step 11 | 60° C. 2 min |
| Step 12 | go to 11 for 9 times |
| Step 13 | 4° C. forever |
| Step 14 | end |

B. First strand cDNA synthesis method 2

| | |
|---|---|
| mRNA (10 μg) | 25 μl |
| water | 30 μl |
| *Not I adapter primer (5 μg) | 10 μl |
| 65° C. for 10 min, chill on ice, then add following reagents, | |
| *5× first buffer | 20 μl |
| *0.1 M DTT | 10 μl |
| *10 mM dNTP mix | 5 μl |

Incubate at 45° C. for 2 min, then add 10 μl of *Superscript II RT (200 u/μl), start the following program:

| | |
|---|---|
| Step 1 | 45° C. for 6 sec, −0.1° C./cycle |
| Step 2 | go to 1 for 99 additional cycles |
| Step 3 | 35° C. for 5 min |
| Step 4 | 45° C. for 60 min |
| Step 5 | 50° C. for 10 min |
| Step 6 | 4° C. forever |
| Step 7 | end |

After the 1st strand cDNA synthesis, the DNA is extracted by phenol according to standard procedures, and then precipitated in NaOAc and ethanol, and stored in −20° C.

C. Oxidization of the diol group of mRNA for biotin labeling

First strand cDNA is spun down and washed once with 70% EtOH. The pellet resuspended in 23.2 μl of DEPC treated water and put on ice. Prepare 100 mM of NaIO4 freshly, and then add the following reagents:

| | |
|---|---|
| mRNA:1st cDNA (start with 20 μg mRNA) | 46.4 μl |
| 100 mM NaIO4 (freshly made) | 2.5 μl |
| NaOAc 3M pH 4.5 | 1.1 μl |

To make 100 mM NaIO4, use 21.39 μg of NaIO4 for 1 μl of water. Wrap the tube in a foil and incubate on ice for 45 min. After the incubation, the reaction is then precipitated in:

| | |
|---|---|
| 5 M NaCl | 10 μl |
| 20% SDS | 0.5 μl |
| isopropanol | 61 μl |

Incubate on ice for at least 30 min, then spin it down at max speed at 4° C. for 30 min and wash once with 70% ethanol and then 80% EtOH.

D. Biotinylation of the mRNA diol group

Resuspend the DNA in 110 μl DEPC treated water, then add the following reagents:

| | |
|---|---|
| 20% SDS | 5 μl |
| 2 M NaOAc pH 6.1 | 5 μl |
| 10 mm biotin hydrazide (freshly made) | 300 μl |

Wrap in a foil and incubate at room temperature overnight.

E. RNase I treatment

| | |
|---|---|
| Precipitate DNA in: | |
| 5 M NaCl | 10 μl |
| 2 M NaOAc pH 6.1 | 75 μl |
| biotinylated mRNA:cDNA | 420 μl |
| 100% EtOH (2.5 Vol) | 1262.5 μl |

(Perform this precipitation in two tubes and split the 420 μl of DNA into 210 μl each, add 5 μl of 5M NaCl, 37.5 μl of 2M NaOAc pH 6.1, and 631.25 μl of 100% EtOH). Store at −20° C. for at least 30 min. Spin the DNA down at 4° C. at maximal speed for 30 min. and wash with 80% EtOH twice, then dissolve DNA in 70 μl RNase free water. Pool two tubes and end up with 140 μl.

Add the following reagents:

| | |
|---|---|
| RNase One 10 U/μl | 40 μl |
| 1st cDNA:RNA | 140 μl |
| 10X buffer | 20 μl |

Incubate at 37° C. for 15 min.

Add 5 μl of 40 μg/μl yeast tRNA to each sample for capturing.

F. Full length 1st cDNA capturing

Blocking the beads with yeast tRNA:

| | | |
|---|---|---|
| Beads | 1 | ml |
| Yeast tRNA 40 μg/μl | 5 | μl |

Incubate on ice for 30 min with mixing, wash 3 times with 1 ml of 2M NaCl, 50 mmEDTA, pH 8.0.

Resuspend the beads in 800 μl of 2M NaCl, 50 mm EDTA, pH 8.0, add RNase I treated sample 200 μl, and incubate the reaction for 30 min at room temperature. Capture the beads using the magnetic stand, save the supernatant, and start following washes:

2 washes with 2 M NaCl, 50 mm EDTA, pH 8.0, 1 ml each time,
1 wash with 0.4% SDS, 50 μg/ml tRNA,
1 wash with 10 mm Tris-Cl pH 7.5, 0.2 mm EDTA, 10 mm NaCl,
20% glycerol,
1 wash with 50 μg/ml tRNA,
1 wash with 1^{st} cDNA buffer

G. Second strand cDNA synthesis

Resuspend the beads in:

| | |
|---|---|
| 5X first buffer | 8 μl |
| 0.1 mM DTT | 4 μl |
| 10 mm dNTP mix | 8 μl |
| 5X 2nd buffer | 60 μl |
| E. Coli Ligase 10U/μl | 2 μl |
| E. Coli DNA polymerase 10U/μl | 8 μl |
| E Coli RNaseH 2U/μl | 2 μl |
| P32 dCTP 10 μci/μl | 2 μl |
| Or water up to 300 μl | 208 μl |

Incubate at 16° C. for 2 hr with mixing the reaction in every 30 min.

Add 4 μl of T4 DNA polymerase and incubate for additional 5 min at 16° C.

Elute 2nd cDNA from the beads.

Use a magnetic stand to separate the 2nd cDNA from the beads, then resuspend the beads in 200 μl of water, and then separate again, pool the samples (about 500 μl),

Add 200 μl of water to the beads, then 200 μl of phenol:chloroform, vortex, and spin to separate the sample with phenol.

Pool the DNA together (about 700 μl) and use phenol to clean the DNA again, DNA is then precipitated in 2 μg of glycogen and 0.5 vol of 7.5M NH4OAc and 2 vol of 100% EtOH. Precipitate overnight. Spin down the pellet and wash with 70% EtOH, air-dry the pellet.

| | | | |
|---|---|---|---|
| DNA | 250 μl | DNA | 200 μl |
| 7.5 M NH4OAc | 125 μl | 7.5 M NH4OAc | 100 μl |
| 100% EtOH | 750 μl | 100% EtOH | 600 μl |
| glycogen 1 μg/μl | 2 μl | glycogen 1 μg/μl | 2 μl |

H. Sal I adapter ligation

Resuspend the pellet in 26 μl of water and use 1 μl for TAE gel.

Set up reaction as following:

| | |
|---|---|
| 2^{nd} strand cDNA | 25 μl |
| 5X T4 DNA ligase buffer | 10 μl |
| Sal I adapters | 10 μl |
| T4 DNA ligase | 5 μl |

Mix gently, incubate the reaction at 16° C. overnight.

Add 2 μl of ligase second day and incubate at room temperature for 2 hrs (optional).

Add 50 μl water to the reaction and use 100 μl of phenol to clean the DNA, 90 μl of the upper phase is transferred into a new tube and precipitate in:

| | |
|---|---|
| Glycogen 1 μg/μl | 2 μl |
| Upper phase DNA | 90 μl |
| 7.5 M NH4OAc | 50 μl |
| 100% EtOH | 300 μl |

precipitate at −20° C. overnight

Spin down the pellet at 4° C. and wash in 70% EtOH, dry the pellet.

I. Not I digestion

| | |
|---|---|
| 2^{nd} cDNA | 41 μl |
| Reaction 3 buffer | 5 μl |
| Not I 15u/μl | 4 μl |

Mix gently and incubate the reaction at 37° C. for 2 hr.

Add 50 μl of water and 100 μl of phenol, vortex, and take 90 μl of the upper phase to a new tube, then add 50 μl of NH40Ac and 300 μl of EtOH. Precipitate overnight at −20° C.

Cloning, ligation, and transformation are performed per the Superscript cDNA synthesis kit.

### Example 6

This example describes cDNA sequencing and library subtraction.

Individual colonies can be picked and DNA prepared either by PCR with M13 forward primers and M13 reverse primers, or by plasmid isolation. cDNA clones can be sequenced using M13 reverse primers.

cDNA libraries are plated out on 22×22 cm² agar plate at density of about 3,000 colonies per plate. The plates are incubated in a 37° C. incubator for 12-24 hours. Colonies are picked into 384-well plates by a robot colony picker, Q-bot (GENETIX Limited). These plates are incubated overnight at 37° C. Once sufficient colonies are picked, they are pinned onto 22×22 cm² nylon membranes using Q-bot. Each membrane holds 9,216 or 36,864 colonies. These membranes are placed onto an agar plate with an appropriate antibiotic. The plates are incubated at 37° C. overnight.

After colonies are recovered on the second day, these filters are placed on filter paper prewetted with denaturing solution for four minutes, then incubated on top of a boiling water bath for an additional four minutes. The filters are then placed on filter paper prewetted with neutralizing solution for four minutes. After excess solution is removed by placing the filters on dry filter papers for one minute, the colony side of the filters is placed into Proteinase K solution, incubated at 37° C. for 40-50 minutes. The filters are placed on dry filter papers to dry overnight. DNA is then cross-linked to nylon membrane by UV light treatment.

Colony hybridization is conducted as described by Sambrook, J., Fritsch, E. F. and Maniatis, T., (in Molecular Cloning: A laboratory Manual, 2nd Edition). The following probes can be used in colony hybridization:

1. First strand cDNA from the same tissue as the library was made from to remove the most redundant clones.

2. 48-192 most redundant cDNA clones from the same library based on previous sequencing data.

3. 192 most redundant cDNA clones in the entire maize sequence database.

4. A Sal-A20 oligo nucleotide: TCG ACC CAC GCG TCC GAA AAA AAA AAA AAA AAA AAA, removes clones containing a poly A tail but no cDNA.

5. cDNA clones derived from rRNA.

The image of the autoradiography is scanned into computer and the signal intensity and cold colony addresses of each colony is analyzed. Re-arraying of cold-colonies from 384 well plates to 96 well plates is conducted using Q-bot.

### Example 7

This example describes identification of the gene from a computer homology search.

Gene identities can be determined by conducting BLAST (Basic Local Alignment Search Tool; Altschul, S. F., et al., (1993) J. Mol. Biol. 215:403-410; see also www.ncbi.nlm.nih.gov/BLAST/) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences are analyzed for similarity to all publicly available DNA sequences contained in the “nr” database using the BLASTN algorithm. The DNA sequences are translated in all reading frames and compared for similarity to all publicly available protein sequences contained in the “nr” database using the BLASTX algorithm (Gish, W. and States, D. J. Nature Genetics 3:266-272 (1993)) provided by the NCBI. In some cases, the sequencing data from two or more clones containing overlapping segments of DNA are used to construct contiguous DNA sequences.

Sequence alignments and percent identity calculations can be performed using the Megalign program of the LASERGENE bioinformatics computing suite (DNASTAR Inc., Madison, Wis.). Multiple alignment of the sequences can be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

### Example 8

This example describes expression of transgenes in monocot cells.

A transgene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML 103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue; Stratagene). Bacterial transformants can be screened by restriction enzyme digestion of plasmid DNA and limited nucleotide sequence analysis using the dideoxy chain termination method (Sequenase DNA Sequencing Kit; U.S. Biochemical). The resulting plasmid construct would comprise a transgene encoding, in the 5′ to 3′ direction, the maize 27 kD zein promoter, a cDNA fragment encoding the instant polypeptides, and the 10 kD zein 3′ region.

The transgene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (Hoechst Ag, Frankfurt, Germany) or equivalent may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton flying disc (Bio-Rad Labs). The particles are then accelerated into the corn tissue with a Biolistic PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### Example 9

This example describes expression of transgenes in dicot cells.

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), SmaI, KpnI and XbaI. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embryos may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene which can be used to facilitate soybean transformation is a transgene composed of the 35S promoter from Cauliflower Mosaic Virus (Odell et al.(1985) Nature 313:810-812), the hygromycin phosphotransferase gene from plasmid pJR225 (from E. coli; Gritz et al.(1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptides and the phaseolin 3′ region can be isolated as a restriction fragment. This fragment can then be inserted into a unique restriction site of the vector carrying the marker gene.

To 50 μL of a 60 mg/mL 1 m gold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 10

This example describes expression of a transgene in microbial cells.

The cDNAs encoding the instant potypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3 aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% NuSieve GTG low melting agarose gel (FMC). Buffer and agarose contain 10 μg/ml ethidium bromide for visualization of the DNA fragment. The fragment can then be purified from the agarose gel by digestion with GELase (Epicentre Technologies) according to the manufacturer's instructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptides are then screened for the correct orientation with respect to the T7 promoter by restriction enzyme analysis.

For high level expression, a plasmid clone with the cDNA insert in the correct orientation relative to the T7 promoter can be transformed into E. coli strain BL21 (DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25°. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One microgram of protein from the soluble fraction of the culture can be separated by SDS-polyacrylamide gel electrophoresis. Gels can be observed for protein bands migrating at the expected molecular weight.

The above examples are provided to illustrate the invention but not to limit its scope. Other variants of the invention will be readily apparent to one of ordinary skill in the art and are encompassed by the appended claims. All publications, patents, patent applications, and computer programs cited herein are hereby incorporated by reference.

What is claimed is:

1. A method of selecting maize inbred lines capable of conferring resistance to root lodging, comprising:
(a) growing inbred lines;
(b) determining differential expression of genes selected from the group consisting of Hsp70, elongation factor 1α (Ef1α), cytochrome P450-dependent monooxygenase (CYP71C2), and an impedance-induced protein, based on expression analysis of mRNA from root tissues of said lines at defined vegetative growth stages; and
(c) selecting those inbred lines wherein gene expression is within parameters which predict desirable root-lodging scores.

2. The method of claim 1 wherein differential gene expression is determined at the vegetative growth stage selected from the group consisting of V8, V9, V10, V11, and V12.

3. A method of selecting maize inbred lines capable of conferring resistance to root lodging, comprising:
(a) growing inbred lines;
(b) determining differential expression of genes selected from the group consisting of Hsp70, elongation factor 1α (Ef1α), cytochrome P450-dependent monooxygenase (CYP71C2), and an impedance-induced protein, based on expression analysis of mRNA from root tissues of said lines at defined vegetative growth stages selected from the group consisting of V8, V9, V10, V11, and V12; and
(c) selecting those inbred lines wherein gene expression is within parameters which predict desirable root-lodging scores.

4. A method of selecting maize inbred lines capable of conferring resistance to root lodging, comprising:
(a) growing inbred lines;
(b) determining differential expression of genes selected from the group consisting of Hsp70, elongation factor 1α (Ef1α), cytochrome P450-dependent monooxygenase (CYP71C2), and an impedance-induced protein, based on expression analysis of mRNA from root tissues of said lines at defined vegetative growth stages; and
(c) selecting those inbred lines wherein gene expression is within parameters which predict morphological traits associated with root-lodging resistance.

5. The method of claim 4 wherein differential gene expression is determined at the vegetative growth stage selected from the group consisting of V8, V9, V10, V11, and V12.

6. A method of selecting maize inbred lines capable of conferring resistance to root lodging, comprising:
(a) growing inbred lines;
(b) determining differential expression of genes selected from the group consisting of Hsp70, elongation factor 1α (Ef1α), cytochrome P450-dependent monooxygenase (CYP71C2), and an impedance-induced protein, based on expression analysis of mRNA from root tissues of said lines at defined vegetative growth stages selected from the group consisting of V8, V9, V10, V11, and V12; and
(c) selecting those inbred lines wherein gene expression is within parameters which predict morphological traits associated with root-lodging resistance.

7. A recombinant expression cassette comprising a polynucleotide operably linked, in sense or anti-sense orientation, to a promoter, wherein said cassette is capable of conferring improved root lodging resistance in a plant transformed with said cassette, and further wherein said polynucleotide is selected from the group consisting of TrpA, Hsp70, elongation factor 1α (Ef1α), cytochrome P450-dependent monooxygenase (CYP71C2), and an impedance-induced protein.

8. A host cell comprising the recombinant expression cassette of claim 7.

9. A transgenic plant comprising a recombinant expression cassette of claim 7.

10. The transgenic plant of claim 9, wherein said plant is a monocot.

11. The transgenic plant of claim 9, wherein said plant is a dicot.

12. The transgenic plant of claim 9, wherein said plant is selected from the group consisting of: maize, soybean, sunflower, sorghum, canola, wheat, alfalfa, cotton, rice, barley, millet, peanut, and cocoa.

13. A transgenic seed from the transgenic plant of claim 9.

14. A method of modulating root quality in a plant, comprising:
(a) introducing into a plant cell a recombinant expression cassette comprising a polynucleotide operably linked to a promoter, wherein said polynucleotide is selected from the group consisting of TrpA, Hsp70, elongation factor 1α (Ef1α), cytochrome P450-dependent monooxygenase (CYP71C2), and an impedance-induced protein;
(b) culturing the plant cell under plant cell growing conditions;
(c) regenerating a plant from said cultured plant cell; and
(d) inducing expression of said polynucleotide for a time sufficient to modulate root quality in said plant.

15. The method of claim 14, wherein the plant is selected from the group consisting of: maize, soybean, sunflower, sorghum, canola, wheat, alfalfa, cotton, rice, barley, millet, peanut, and cocoa.

16. A method of modulating root lodging resistance in a plant, comprising:
(a) introducing into a plant cell a recombinant expression cassette comprising a polynucleotide operably linked to a promoter, wherein said polynucleotide is selected from the group consisting of TrpA, Hsp70, elongation factor 1α (Ef1α), cytochrome P450-dependent monooxygenase (CYP71C2), and an impedance-induced protein;
(b) culturing the plant cell under plant cell growing conditions;
(c) regenerating a plant from said cultured plant cell; and
(d) inducing expression of said polynucleotide for a time sufficient to modulate root lodging resistance in said plant.

17. The method of claim 16, wherein the plant is selected from the group consisting of: maize, soybean, sunflower, sorghum, canola, wheat, alfalfa, cotton, rice, barley, millet, peanut, and cocoa.

18. A method of selecting maize inbred lines capable of conferring resistance to root lodging, comprising:
(a) growing inbred lines which differ in root-lodging phenotype;
(b) identifying a TrpA polymorphism associated with root-lodging resistance;
(c) screening candidate maize lines for existence of the identified polymorphism.

19. The method of claim 18 wherein the TrpA polymorphism is the same as the polymorphism in SEQ ID No. 1 or SEQ ID No. 3.
